(19) United States
(12) Patent Application Publication
(10) Pub. No.: US 2010/0234739 A1
(43) Pub. Date: Sep. 16, 2010

(54) SPECTROSCOPIC OBSERVATION DEVICE, ENDOSCOPE SYSTEM, AND CAPSULE ENDOSCOPE SYSTEM

(75) Inventors: Masaya NAKAOKA, Tokyo (JP); Yasuhiro KAMIHARA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/783,820

(22) Filed: May 20, 2010

Related U.S. Application Data
(63) Continuation of application No. PCT/JP2007/073337, filed on Dec. 3, 2007.

(51) Int. Cl. A61B 6/00; A61B 1/06

(52) U.S. Cl. 600/476; 600/160

(57) ABSTRACT

A spectroscopic observation device enables proper observation by respectively meeting the observation condition where easy-to-observe image with high S/N ratio is preferable and the observation condition where it is preferable to restrain interfusion of any other fluorescent components, even when fluorescence of the same wavelength is to be observed. The spectroscopic observation device comprises: an excitation light source (8) to irradiate excitation light toward an observation target; a spectroscopic element (12) which can separate fluorescence emitted out of the observation target by the irradiation of the excitation light coming from the excitation light source (8), into a plurality of types of fluorescence wavebands having the same center wavelength and different pass bands; and an image pickup section (13) which takes an image of the fluorescence separated by the spectroscopic element (12).

### CROSS REFERENCE TO RELATED APPLICATIONS

This is a continuation of International Application PCT/JP/2007/073337, with an international filing date of Dec. 3, 2007, which is hereby incorporated by reference herein in its entirety.

### TECHNICAL FIELD

The present invention relates to a spectroscopic observation device, an endoscope system, and a capsule endoscope system.

### BACKGROUND ART

Conventionally, there is known an endoscope apparatus for fluorescence spectroscopy capable of separating fluorescence wavelengths by using a spectroscopic element (for example, refer to Patent Citation 1).

With this endoscope apparatus for fluorescence spectroscopy, it is possible to observe fluorescence generated by the excitation of a fluorescent agent which has been administered into or scattered inside an organism body, as well as to observe fluorescence generated by the excitation of a fluorescent substance which has originally existed in the organism body, that is to say, autofluorescence.

Patent Citation 1:

Japanese Unexamined Patent Application, Publication No. 2006-25802

### DISCLOSURE OF INVENTION

In particular, such wavelength separation and imaging of fluorescence from an observation target within an organism body involves a disadvantage in that, the applicable waveband of drug fluorescence is relatively limited because of a property in which short-wavelength light is easily scattered and thus is difficult to transmit inside the organism body while long-wavelength light is easily absorbed by moisture and thus is also difficult to transmit inside the organism body.

For this reason, the use of a plurality of fluorescent agents has a tendency in that fluorescent components are easily interfused because their excitation wavelengths and their fluorescence wavelengths are close to each other.

In addition, the observation of autofluorescence is also considered to have the same tendency because a plurality of autofluorescent substances may emit fluorescence by a single excitation light wavelength as various autofluorescent components do exist inside an organism body.

Moreover, since fluorescence intensities are generally weak, fluorescence tends to be vulnerable to readout noise of an image pickup element and other noise components such as a dark current.

The present invention was made to address the above-mentioned situations with an object of providing a spectroscopic observation device and an endoscope system, with which proper observation can be performed by respectively meeting the observation condition where easy-to-observe image with high S/N ratio is preferable and the observation condition where it is preferable to restrain interfusion of any other fluorescent components, even when fluorescence of the same wavelength is to be observed.

In order to achieve the above-mentioned object, the present invention provides the following solutions.

A first aspect of the present invention is a spectroscopic observation device comprising: an excitation light source to irradiate excitation light toward an observation target; a spectroscopic element which can separate fluorescence emitted out of the observation target by the irradiation of the excitation light coming from the excitation light source, into a plurality of types of fluorescence wavebands having the same center wavelength and different pass bands; and an image pickup section which takes an image of the fluorescence separated by the spectroscopic element.

In the above-mentioned first aspect, the spectroscopic element may also comprise a variable spectroscopic element which has a plurality of optical members arranged to face to each other across a space and an actuator to change the space between these optical members.

In addition, in the above-mentioned first aspect, the structure may also be such that: the spectroscopic element comprises two or more types of optical filters having different transmittance characteristics to transmit light with the same center wavelength and different pass bands; the image pickup section comprises an image pickup element having a plurality of pixels arranged in two-dimension; and the optical filter and the image pickup element are arranged so that fluorescence transmitted through different optical filters can be imaged on different pixels of the image pickup element.

In addition, the above-mentioned structure may also be such that the image pickup section comprises two or more image pickup elements and a beam splitter which splits fluorescence emitted from the observation target into beams respectively traveling toward the image pickup elements; and the two or more types of optical filters having different transmittance characteristics are arranged to face different image pickup elements.

Moreover, in the above-mentioned first aspect, the spectroscopic observation device may also comprise a mode setting section which selectively sets either a first imaging mode for taking an image of fluorescence in a first waveband and a second imaging mode for taking an image of fluorescence in a second waveband whose pass band is narrower than that of the first waveband.

In addition, in the above-mentioned structure, the mode setting section may set the first imaging mode prior to the second imaging mode.

A second aspect of the present invention is an endoscope system comprising the above-mentioned spectroscopic observation device.

A third aspect of the present invention is a capsule endoscope system comprising the above-mentioned spectroscopic observation device.

The present invention offers an effect of enabling proper observation by respectively meeting the observation condition where easy-to-observe image with high S/N ratio is preferable and the observation condition where it is preferable to restrain interfusion of any other fluorescent components, even when fluorescence of the same wavelength is to be observed.

### BRIEF DESCRIPTION OF DRAWINGS

FIG. 1 is a block diagram showing the overall structure of an endoscope system according to a first embodiment of the present invention.

FIG. 2 is a schematic structure diagram showing the inner structure of an image pickup unit of the endoscope system of FIG. 1.

FIG. 3 shows transmittance characteristics of respective optical members constructing the endoscope system of FIG. 1, and wavelength characteristics of irradiation light and fluorescence.

FIG. 4 is a schematic diagram showing the inner structure of an image pickup unit of an endoscope system according to a second embodiment of the present invention.

FIG. 5 shows filters provided in the image pickup unit of FIG. 4.

FIG. 6 is a schematic diagram showing the structure of a capsule endoscope system according to a third embodiment of the present invention.

FIG. 7 is a schematic diagram showing the inner structure of an image pickup unit of an endoscope system according to a fourth embodiment of the present invention.

FIG. 8A shows filters provided in the image pickup unit of FIG. 7.

FIG. 8B shows filters provided in the image pickup unit of FIG. 7.

### EXPLANATION OF REFERENCE

A: Observation target
1: Endoscope system
8: Excitation light source
12: Variable spectroscopic element (spectroscopic element)
12a and 12b: Optical members
12c: Actuator
13, 22a, and 22b: Image pickup elements (image pickup section)
16: Change-over switch (mode setting section)
20a: First filter (optical filter)
20b: Second filter (optical filter)
20c: Third filter (optical filter)
20d: Fourth filter (optical filter)
21: Beam splitter

### BEST MODE FOR CARRYING OUT THE INVENTION

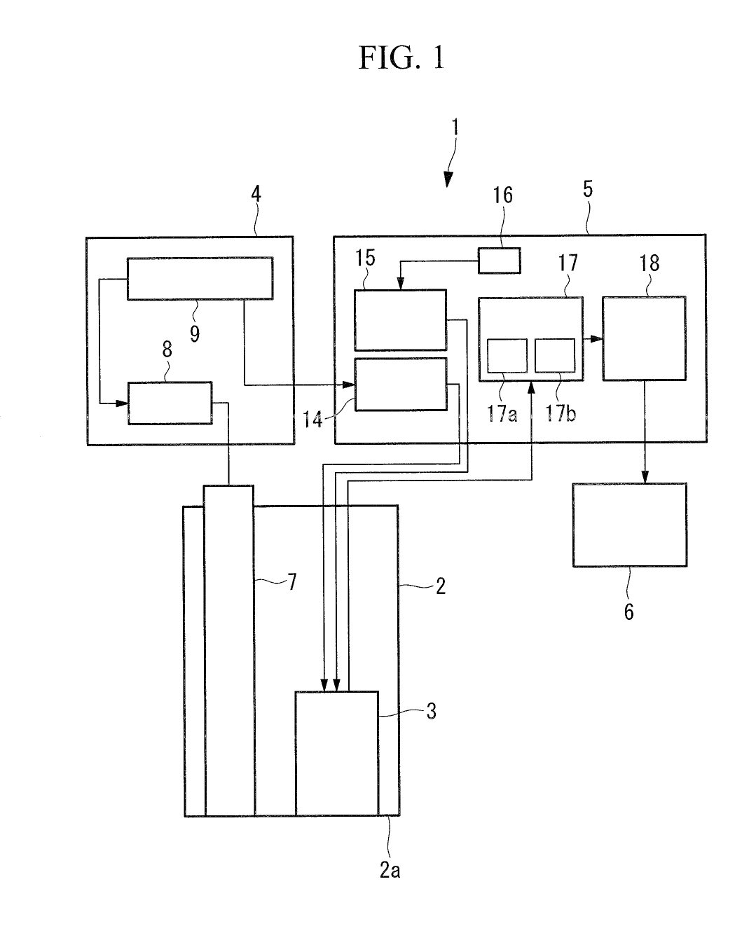

Hereunder is a description of a spectroscopic observation device and an endoscope system according to a first embodiment of the present invention, with reference to FIG. 1 to FIG. 3.

The spectroscopic observation device according to this embodiment is equipped in the endoscope system 1 shown in FIG. 1.

As shown in FIG. 1, the endoscope system 1 according to this embodiment comprises: an inserting unit 2 to be inserted in a body cavity of an organism; an image pickup unit 3 disposed in the inserting unit 2; a light source unit 4 which emits excitation light; a control unit 5 which controls the image pickup unit 3 and the light source unit 4; and a display unit 6 which displays an image acquired by the image pickup unit 3.

In addition, the spectroscopic observation device according to this embodiment comprises the image pickup unit 3, the light source unit 4, and the control unit 5.

The inserting unit 2 has a very thin outer-dimension to be inserted into a body cavity of an organism, and comprises therein the image pickup unit 3 and a light guide 7 which propagates light from the light source unit 4 to the distal end 2a.

The light source unit 4 comprises: an excitation light source 8 which emits excitation light to irradiate an observation target in the body cavity and to excite a fluorescent substance existing in the observation target A to thereby generate fluorescence; and a light source control circuit 9 which controls the excitation light source 8.

The excitation light source 8 is, for example, a semiconductor laser which emits excitation light having a peak wavelength of 660±5 nm. The excitation light of such a wavelength can excite fluorescent agents such as Cy5.5 (registered trademark of GE Healthcare, Inc. (formerly Amersham Biosciences Corp.)) and Alexa Fluor (registered trademark) 700 (of Molecular Probes, Inc.).

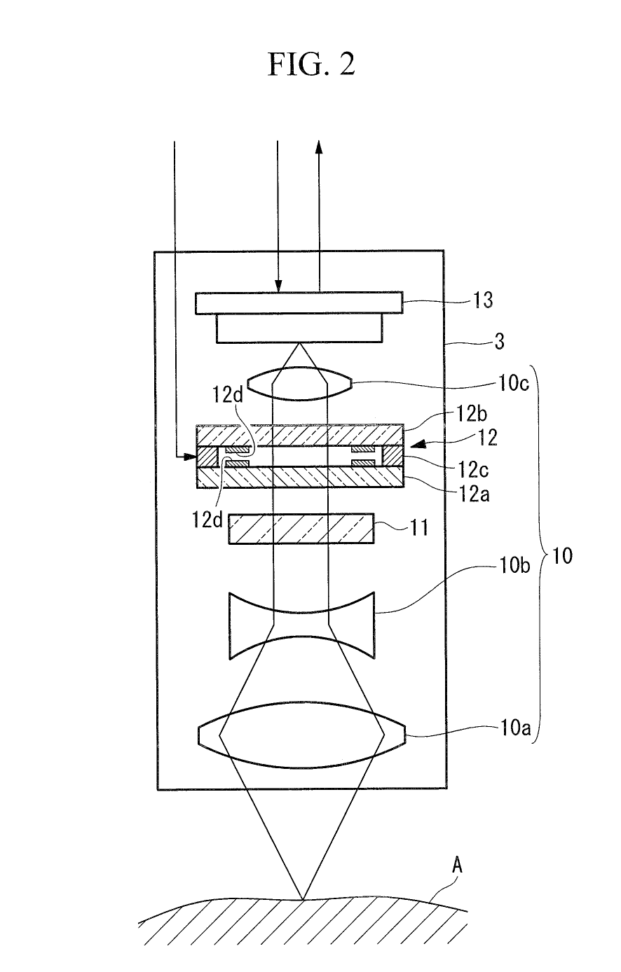

As shown in FIG. 2, the image pickup unit 3 comprises: an image pickup optical system 10 for condensing light incident from the observation target A; an excitation light cut-off filter 11 which cuts-off excitation light incident from the observation target A; a variable spectroscopic element (variable spectroscopy section) 12 which can change spectral characteristics by the operation of the control unit 5; and an image pickup element (image pickup section) 13 which captures the light condensed by the image pickup optical system 10 and converts the light into an electrical signal. The reference sign 10a denotes a condenser lens, the reference sign 10b denotes a collimate lens, and the reference sign 10c denotes an imaging lens.

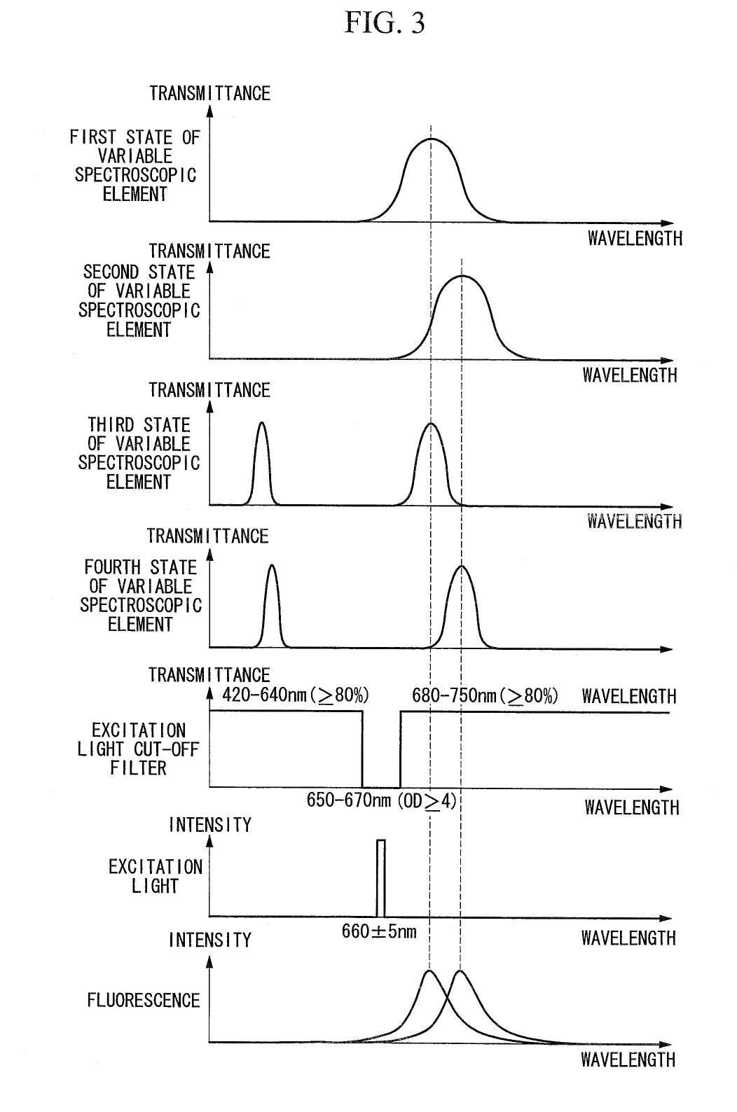

As shown in FIG. 3, the excitation light cut-off filter 11 has transmittance characteristics such that the transmittance for light in a waveband from 420 nm to 640 nm is 80% or higher, the OD value for light in a waveband from 650 nm to 670 nm is 4 or higher (=the transmittance of 1×10⁻⁴ or lower), and the transmittance for light in a waveband from 680 nm to 750 nm is 80% or higher.

The variable spectroscopic element 12 is an etalon-type optical filter comprising: two planar optical members 12a and 12b arranged in parallel across a space and respectively provided with reflection films (not shown) on their facing surfaces; and an actuator 12c to change the space between the Optical members 12a and 12b. The actuator 12c is, for example, a piezoelectric element. This variable spectroscopic element 12 changes the space dimension between the optical members 12a and 12b by the operation of the actuator 12c, thereby changing the waveband of the transmission light.

The space dimension between the optical members 12a and 12b is set at a minute value, for example, in micron or smaller order.

In addition, the actuator has a stroke determined by the following relational expression:

S≧(m₂₋m₁)λ₀/(2n·cos θ),

where m₁ and m₂ refer to orders of interference (m₂>m₁), S refers to the stroke, λ₀ refers to the transmission wavelength, n refers to the refractive index between the optical members 12a and 12b, and θ refers to the incident angle of light entering the space the optical members 12a and 12b.

Further, ring-shaped capacitance sensor electrodes 12d are arranged outside the optically effective diameters of the optical members 12 and 12b.

The reflection films are made of, for example, dielectric multilayer films.

Furthermore, the capacitance sensor electrodes 12d are made of metallic films. Signals from the capacitance sensor electrodes 12d are fed back so as to control a drive signal to the actuator 12c, thereby improving the adjusting precision of the transmittance characteristic.

In this embodiment, the variable spectroscopic element 12 has a variable pass band within a waveband including wavelengths of two types of fluorescence (drug fluorescence) emitted from fluorescent agents by the excitation of excitation light (for example, from 680 nm to 740 nm). In addition, the variable spectroscopic element 12 can be changed into four states in accordance with the control signal from the control unit 5.

The first state is a state in which the pass band within the variable pass band is set between 680 nm and 720 nm in terms of full width at half maximum to thereby transmit fluorescence from Cy5.5.

The second state is a state in which the pass band within the variable pass band is set between 700 nm and 740 nm in terms of full width at half maximum to thereby transmit fluorescence from Alexa Fluor 700.

The third state is a state in which the pass band within the variable pass band is set between 690 nm and 710 nm in terms of full width at half maximum to thereby transmit fluorescence from Cy5.5 likewise of the first state.

The fourth state is a state in which the pass band within the variable pass band is set between 710 nm and 730 nm in terms of full width at half maximum to thereby transmit fluorescence from Alexa Fluor 700 likewise of the second state.

The first state and the second state serve as a state (first imaging mode) in which the pass band regarding a same order of interference of the variable spectroscopic element 12 is adjusted so that the pass band can match to the wavebands of fluorescence from two types of fluorescent agents.

The third state and the fourth state serve as a state (second imaging mode) in which the pass band regarding another order differing from the order of the first state and the second state is matched to the wavebands of fluorescence from these two types of fluorescent agents.

In addition, the first state and the third state, or the second state and the fourth state, respectively serve as states in which the pass bands of different orders of interference of the variable spectroscopic element 12 are matched to the waveband of same drug fluorescence. The pass band of a smaller order is broader than the pass band of a greater order, and is capable of transmitting fluorescence in a broader waveband. On the other hand, the narrower pass band of a greater order is capable of transmitting fluorescence in a narrower waveband.

As shown in FIG. 1, the control unit 5 comprises: an image pickup element drive circuit (image pickup element control circuit) 14 which controls the driving of the image pickup element 13; a variable spectroscopic element control circuit 15 which controls the driving of the variable spectroscopic element 12; a change-over switch (mode setting section) 16 which is connected to the variable spectroscopic element control circuit 15 and is operated by an operator; a frame memory 17 which stores image information acquired by the image pickup element 13; and an image processing circuit 18 which processes the image information stored in the frame memory 17 and outputs the processed information to the display unit 6.

The image pickup element drive circuit 14 is connected to the light source control circuit 9 to control the driving of the image pickup element 13 synchronously with the operation of the excitation light source 8 done by the light source control circuit 9.

The change-over switch 16 is for example a switch to select a state from the above-mentioned four states of the variable spectroscopic element 12. When any one of the first to fourth states is selected by the change-over switch 16, a voltage for executing the selected state of the first to fourth states is supplied from the variable spectroscopic element control circuit 15 to the variable spectroscopic element 12 so that the variable spectroscopic element 12 can be set according to the voltage to the concerned state of the first to fourth states.

The frame memory 17 comprises a first frame memory 17a and a second frame memory 17b so that, for example, the image information acquired by the image pickup element 13 can be stored in the first frame memory 17a when the variable spectroscopic element 12 is in the first or second state and the image information acquired by the image pickup element 13 can be stored in the second frame memory 17b when the variable spectroscopic element 12 is in the third or fourth state.

Moreover, the image processing circuit 18 is designed, for example, to output the image information received from the first frame memory 17a to a first channel of the display unit 6 and to output the image information received from the second frame memory 17b to a second channel of the display unit 6.

Hereunder is a description of the operation of the thus constructed endoscope system 1 according to this embodiment.

In order to take an image of the observation target A in a body cavity of an organism by using the endoscope system 1 according to this embodiment, the inserting unit 2 is inserted into the body cavity while injecting the fluorescent agents into the body, and then the distal end 2a thereof is located to face the observation target A in the body cavity. In this state, the light source unit 4 and the control unit 5 are operated so that the excitation light source 8 can be turned on to generate excitation light by the operation of the light source control circuit 9.

The excitation light generated in the light source unit 4 is propagated through the light guide 7 to the distal end 2a of the inserting unit 2, and then irradiated from the distal end 2a of the inserting unit 2 onto the observation target A.

When the excitation light is irradiated on the observation target A, the fluorescent agents existing in the observation target A are excited to emit fluorescence. The fluorescence generated from the observation target A is transmitted through the condenser lens 10a, the collimate lens 10b, and the excitation light cut-off filter 11 of the image pickup unit 3 to be incident into the variable spectroscopic element 12.

Since the state of the variable spectroscopic element 12 is switchable by the operation of the variable spectroscopic element control circuit 15 so as to comply with the operation of the change-over switch 16 by the operator, it is possible to transmit fluorescence in the pass band corresponding to the selected state, out of the incident light. In this case, a part of the excitation light irradiated on the observation target A is reflected by the observation target A and is made incident into the image pickup unit 3 together with the fluorescence. However, since the excitation light cut-off filter 11 is provided in the image pickup unit 3, that excitation light can be blocked and thereby prevented from entering the image pickup element 13.

Then, the fluorescence transmitted through the variable spectroscopic element 12 is made incident into the image pickup element 13, by which image information (fluorescence image) is acquired. The acquired image information is stored in the first or second frame memory 17a or 17b in accordance with the selected state of the variable spectroscopic element, and then is output to the first or second channel of the display unit 6 by the image processing circuit 18 to be displayed on the display unit 6.

That is to say, in order to observe fluorescence from Cy5.5, the operator operates the change-over switch 16 to select the first state or the third state. In this case, the first state is selected if it is preferable to acquire a bright fluorescence image while the third state is selected if it is preferable to precisely perform spectral separation from other wavelengths. By selecting the third state, the pass band width can be narrowed down without moving the center wavelength of the pass band from the first state. Therefore, interfusion with fluorescence of unneeded wavelengths can be prevented although the intensity of the acquired image is weakened.

In addition, in order to observe fluorescence from Alexa Fluor 700, the operator operates the change-over switch 16 to select the second state or the fourth state. In this case, similarly to the above-mentioned case, the second state is selected if it is preferable to acquire a bright fluorescence image while the fourth state is selected if it is preferable to precisely perform spectral separation from other wavelengths. By selecting the fourth state, the pass band width can be narrowed down without moving the center wavelength of the pass band from the second state. Therefore, interfusion with fluorescence of unneeded wavelengths can be prevented.

In this case, according to this embodiment, the variable spectroscopic element 12 can be changed from the first state to the third state, or from the second state to the fourth state, respectively, simply by moving the actuator 12c by the stroke S which is determined by the following equation:

S=λ₀/(2n·cos θ).

By so doing, it is easily possible, on demand, to select and transmit fluorescence among a plurality of types of wavebands having the same center wavelength and different pass bands.

According to the endoscope system 1 of this embodiment, during the process for inserting the inserting unit 2 into the body cavity and moving the distal end 2a closer to the observation target A, it is preferable to firstly set the variable spectroscopic element 12 to the first or second state prior the third or fourth state. When the distal end 2a of the inserting unit 2 is apart from the observation target A, it is possible, by setting the variable spectroscopic element 12 to the first state or the second state, to transmit as large quantity of fluorescence as possible from the observation target A. Moreover, when the distal end 2a of the inserting unit 2 is located close to the observation target A, it is possible, by setting the variable spectroscopic element 12 to the third state or the fourth state, to acquire an image of highly precisely separated fluorescence with less interfusion of any other fluorescent components.

Moreover, in this embodiment, the description was made concerning the case where the pass band width is changed by incrementing or reducing one order of interference of the variable spectroscopic element 12, although two or more orders of interference may be incremented or reduced.

In this case, the actuator 12c can be moved by the stroke S which is determined by the following equation:

S=dλ₀/(2n·cos θ),

where d refers to the incremented or reduced number of the order of interference.

Moreover, in this embodiment, the variable spectroscopic element 12 is set from the first state to the fourth state by the selection of the operator. However, instead of this, it is also possible such that the operator is allowed to select either the first imaging mode for taking images by alternately switching over between the first sate and the second state at predetermined timings, or the second imaging mode for taking images by alternately switching over between the third state and the fourth state at predetermined timings. In the first imaging mode, both a bright fluorescence image of Cy5.5 and a bright fluorescence image of Alexa Fluor 700 can be acquired and displayed at the same time. Moreover, in the second imaging mode, both a highly precisely separated fluorescence image of Cy5.5 and a highly precisely separated fluorescence image of Alexa Fluor 700 can be acquired and displayed at approximately the same time.

Furthermore, in this embodiment, the imaging mode is switched over by the selection of the operator. However, instead of this, automatic selection can also be employed in such a way that, for example, the image information acquired by the image pickup element 13 is processed to thereby extract light quantity information, and then, if the light quantity is determined to be insufficient, the first imaging mode is selected, or, if the light quantity is sufficient, the second imaging mode is selected.

Alternatively, it is also possible such that the operator is allowed to select either an imaging mode for taking fluorescence images of Cy5.5 or an imaging mode for taking fluorescence images of Alexa Fluor 700, and in each imaging mode, the variable spectroscopic element 12 can be alternately switched over between the first state and the third state, or between the second state and the fourth state, at predetermined timings. By so doing, a bright fluorescence image and a highly precisely separated fluorescence image of either Cy5.5 or Alexa Fluor 700 can be acquired and displayed at approximately the same time.

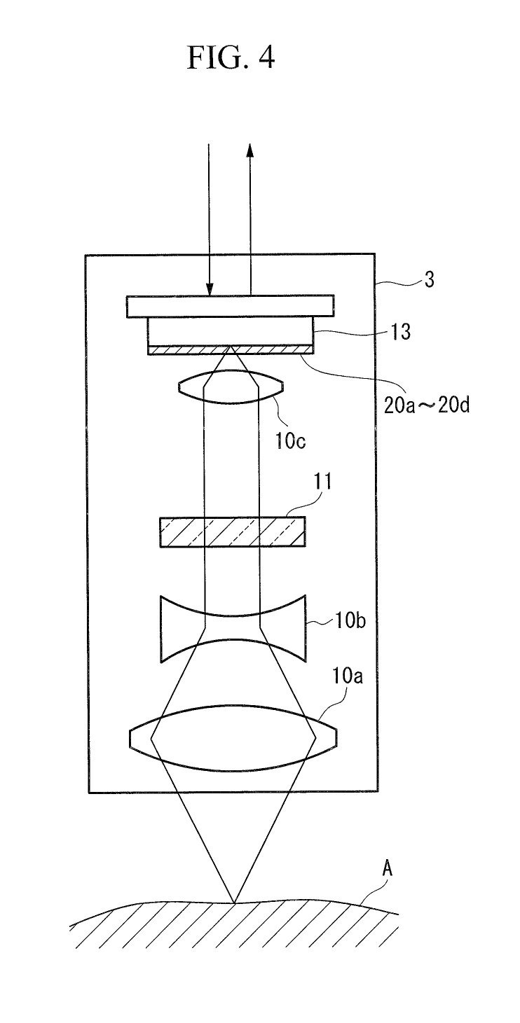

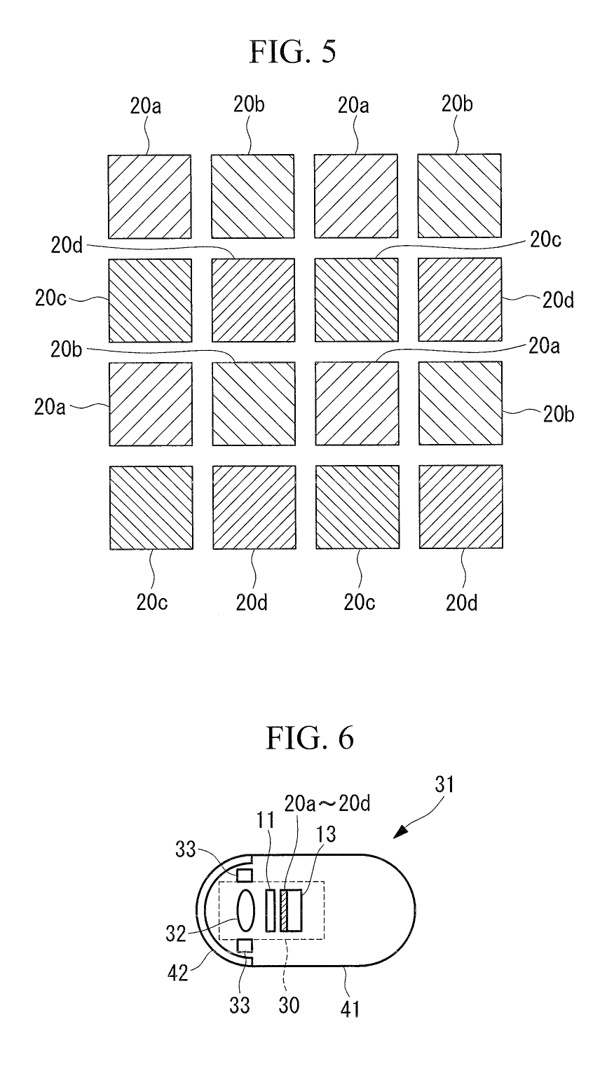

Next is a description of a spectroscopic observation device and an endoscope system according to a second embodiment of the present invention, with reference to FIG. 4 and FIG. 5.

In the following description of this embodiment, parts having common structures to those of the spectroscopic observation device and the endoscope system according to the above-mentioned first embodiment are denoted by the same reference signs, and are not described.

In the spectroscopic observation device and the endoscope system according to this embodiment, instead of the variable spectroscopic element 12 of the first embodiment, four types of filters (optical filters) 20a to 20d having the following pass bands are arranged in a mosaic shape to correspond to respective pixels of the image pickup element 13.

Pass band of the first filter: from 680 nm to 720 nm in terms of full width at half maximum

Pass band of the second filter: from 700 nm to 740 nm in terms of full width at half maximum

Pass band of the third filter: from 690 nm to 710 nm in terms of full width at half maximum

Pass band of the fourth filter: from 710 nm to 730 nm in terms of full width at half maximum

According to the thus constructed spectroscopic observation device and endoscope system of this embodiment, images of fluorescence from 680 nm to 720 nm, from 700 nm to 740 nm, from 690 nm to 710 nm, and from 710 nm to 730 nm, in terms of full width at half maximum, can be respectively acquired from pixels corresponding to the first to fourth filters 20a to 20d.

In addition, similarly to the first embodiment, it is also possible such that the operator is allowed to select either a first imaging mode for taking an image from pixels corresponding to the first and second filters 20a and 20b, or a second imaging mode for taking an image from pixels corresponding the third and fourth filters 20c and 20d, according to the situation. Alternatively, automatic selection can also be employed in such a way that respective image is processed, and then, if the light quantity is determined to be insufficient, the first imaging mode is selected, or, if the light quantity is sufficient, the second imaging mode is selected.

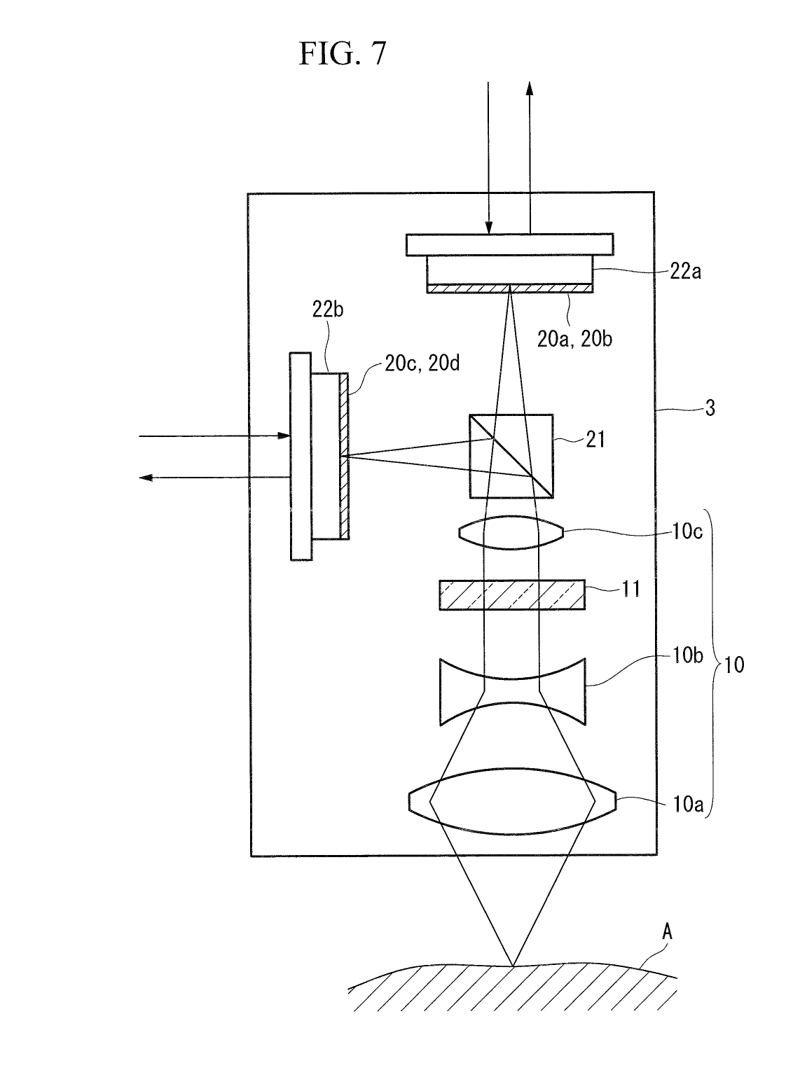

Next is a description of a spectroscopic observation device and a capsule endoscope system according to a third embodiment of the present invention, with reference to FIG. 6. In the following description of this embodiment, parts having common structures to those of the spectroscopic observation device and the endoscope system according to the above-mentioned second embodiment are denoted by the same reference signs, and are not described.

In the spectroscopic observation device and the endoscope system according to this embodiment, the inserting unit 3 of the endoscope system 1 is formed in a capsule shape. Similarly to the second embodiment, four types of filters 20a to 20d are arranged in a mosaic shape to correspond to respective pixels of the image pickup element 13.

In the capsule endoscope 31, an image pickup unit 30 and light emitting elements 33 are disposed inside a transparent cover 42 and a case 41. The image pickup unit 30 comprises a lens 32, the excitation light cut-off filter 11, the filters 20a to 20d, and the image pickup element 13. Similarly to the excitation light source of the first embodiment, the light emitting element 33 is, for example, a semiconductor laser which emits excitation light having a peak wavelength of 660±5 nm. In addition, such a semiconductor laser may be replaced by LED.

Similarly to the second embodiment, it is also possible such that the operator is allowed to select either a first imaging mode for taking an image from pixels corresponding to the first and second filters 20a and 20b, or a second imaging mode for taking an image from pixels corresponding the third and fourth filters 20c and 20d, according to the situation.

Since mosaic-shaped filters are employed instead of the variable spectroscopic element, no variable device is needed. Therefore, considering that capsule endoscopes involve severe spatial restrictions, remarkable effects are given such that the space needing for the variable device can be saved to thereby reduce the size of the capsule endoscope, or to deposit other components in the thus saved space. Furthermore, since no variable device is used, another effect is also given such that the power consumption of the capsule endoscope, to which only a limited power can be supplied, can be saved.

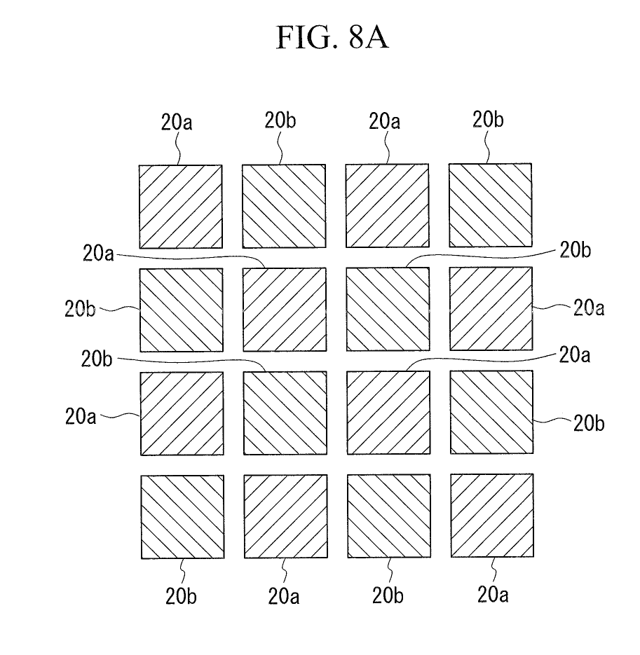

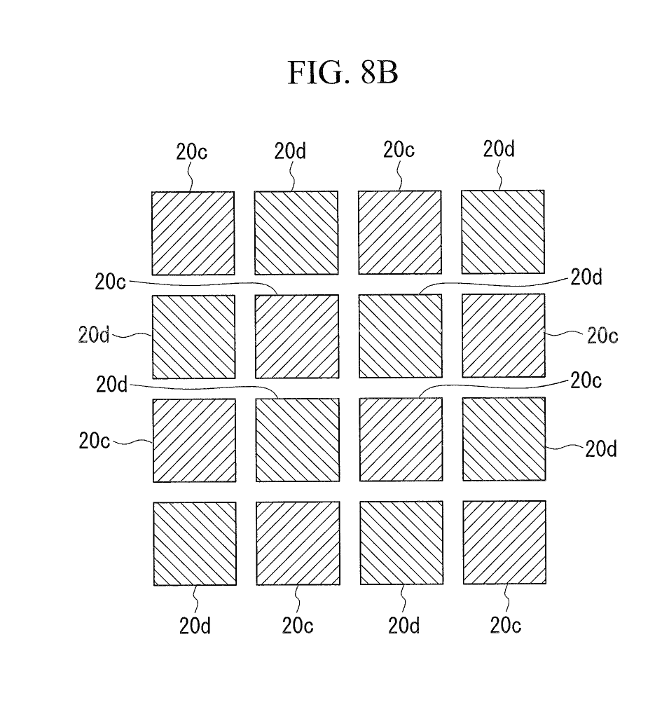

Next is a description of a spectroscopic observation device and an endoscope system according to a fourth embodiment of the present invention, with reference to FIG. 7, FIG. 8A, and FIG. 8B.

In the following description of this embodiment, parts having common structures to those of the spectroscopic observation device and the endoscope system according to the above-mentioned second embodiment are denoted by the same reference signs, and are not described.

The spectroscopic observation device and the endoscope system according to this embodiment comprises: a beam splitter 21 which splits a light beam from the observation target A into two beams; and two image pickup elements 22a and 22b which respectively captures the light beams that have been split by the beam splitter 21. The first and second filters 20a and 20b shown in FIG. 8A are disposed in front of the image pickup element 22a on one side, while the third and fourth filters 20c and 20d shown in FIG. 8B are disposed in front of the image pickup element 22b on another side.

By having such a structure, similarly to the second embodiment, images of fluorescence from 680 nm to 720 nm, from 700 nm to 740 nm, from 690 nm to 710 nm, and from 710 nm to 730 nm, in terms of full widths at half maximum, can be respectively acquired from outputs of the image pickup elements 22a and 22b.

1. A spectroscopic observation device comprising:
an excitation light source to irradiate excitation light toward an observation target;
a spectroscopic element to separate fluorescence emitted out of the observation target by the irradiation of the excitation light coming from the excitation light source, into a plurality of types of fluorescence wavebands having the same center wavelength and different pass bands; and
an image pickup section which takes an image of the fluorescence separated by the spectroscopic element.

2. A spectroscopic observation device according to claim 1, wherein said spectroscopic element comprises a variable spectroscopic element which has a plurality of optical members arranged to face to each other across a space and an actuator to change the space between these optical members.

3. A spectroscopic observation device according to claim 1, wherein said spectroscopic element comprises two or more types of optical filters having different transmittance characteristics to transmit light with the same center wavelength and different pass bands;
said image pickup section comprises an image pickup element having a plurality of pixels arranged in two-dimension; and
said optical filter and said image pickup element are arranged so that fluorescence transmitted through different optical filters can be imaged on different pixels of said image pickup element.

4. A spectroscopic observation device according to claim 3, wherein said image pickup section comprises two or more image pickup elements and a beam splitter which splits fluorescence emitted from said observation target into beams respectively traveling toward said image pickup elements; and
said two or more types of optical filters having different transmittance characteristics are arranged to face different image pickup elements.

5. A spectroscopic observation device according to claim 1, wherein said spectroscopic observation device comprises a mode setting section which selectively sets either a first imaging mode for taking an image of fluorescence in a first waveband and a second imaging mode for taking an image of fluorescence in a second waveband whose pass band is narrower than that of the first waveband.

6. A spectroscopic observation device according to claim 5, wherein said mode setting section sets the first imaging mode prior to said second imaging mode.

7. An endoscope system comprising the spectroscopic observation device according to claim 1.

8. A capsule endoscope system comprising the spectroscopic observation device according to claim 1.
